SEQUENCE LISTING (18 sequences)

SEQ ID NO: 1 - DNA, 143 bp, Mus musculus
  Methanol-responsive region derived from mouse tyrosine hydroxylase gene
attagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgt

SEQ ID NO: 2 - DNA, 100 bp, Mus musculus
  Core promoter region derived from mouse tyrosine hydroxylase gene
aggaggtgggggacccagaggggctttgacgtcagcctggcctttaagaggccgcctgcctggcaagggctgtggagacagaactcgggaccaccagctt

SEQ ID NO: 3 - DNA, 93 bp, Simian virus 40
  Simian virus 40 early promoter
atttatgcagaggccgaggccgcctcggcctctgagctattccagaagtagtgaggaggcttttttggaggcctaggcttttgcaaaaagctt

SEQ ID NO: 4 - DNA, 165 bp, Simian virus 40
  Simian virus 40 late promoter
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgc

SEQ ID NO: 5 - DNA, 758 bp, human herpesvirus 1
  Human herpesvirus 1 thymidine kinase promoter
gatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaa

SEQ ID NO: 6 - DNA, 260 bp, Artificial Sequence
  Methanol-responsive promoter comprising a methanol-responsive region derived from transcription regulatory sequence of mouse tyrosine hydroxylase gene and a core promoter region derived from transcription regulatory sequence of mouse tyrosine hydroxylase gene
attagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtgctagcccgggctcgagaggaggtgggggacccagaggggctttgacgtcagcctggcctttaagaggccgcctgcctggcaagggctgtggagacagaactcgggaccaccagctt

SEQ ID NO: 7 - DNA, 373 bp, Artificial Sequence
  Methanol-responsive promoter comprising a methanol-responsive region derived from transcription regulatory sequence of mouse tyrosine hydroxylase gene and a core promoter region derived from Simian Virus 40 early promoter
attagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagctt

SEQ ID NO: 8 - DNA, 570 bp, Artificial Sequence
  Methanol-responsive promoter comprising three copies of methanol-responsive regions derived from transcription regulatory sequence of mouse tyrosine hydroxylase gene and a core promoter region derived from transcription regulatory sequence of mouse
attagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtgctagcggtaccattagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtgctagcggtaccattagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtgctagcccgggctcgagaggaggtgggggacccagaggggctttgacgtcagcctggcctttaagaggccgcctgcctggcaagggctgtggagacagaactcgggaccaccagctt

SEQ ID NO: 9 - DNA, 34 bp, Artificial Sequence
  Forward primer
cgtggtaccacatacactggggcagtgagtagat

SEQ ID NO: 10 - DNA, 34 bp, Artificial Sequence
  Reverse primer
gcagctagcaagctggtggtcccgagttctgtct

SEQ ID NO: 11 - DNA, 34 bp, Artificial Sequence
  Forward primer
cggggtaccagatttatttgtctccaagggctat

SEQ ID NO: 12 - DNA, 34 bp, Artificial Sequence
  Forward primer
cggggtaccattagagagctctagatgtctcctg

SEQ ID NO: 13 - DNA, 34 bp, Artificial Sequence
  Forward primer
cccggtaccctaatgggacggaggcctctctcgt

SEQ ID NO: 14 - DNA, 34 bp, Artificial Sequence
  Forward primer
cggggtaccgtgggggacccagaggggctttgac

SEQ ID NO: 15 - DNA, 34 bp, Artificial Sequence
  Forward primer
cggctcgaggtgggggacccagaggggctttgac

SEQ ID NO: 16 - DNA, 34 bp, Artificial Sequence
  Forward primer
gccgctagcacgagagaggcctccgtcccattag

SEQ ID NO: 17 - DNA, 34 bp, Artificial Sequence; identical to SEQ ID NO: 12
  Primer for amplifying methanol-responsive region

SEQ ID NO: 18 - DNA, 34 bp, Artificial Sequence; identical to SEQ ID NO: 16
  Primer for amplifying methanol-responsive region

(19) United States
(12) Patent Application Publication
(10) Pub. No.: US 2009/0253167 A1
(43) Pub. Date: Oct. 8, 2009

(54) Methanol-responsive promoter, fusion gene comprising promoter and foreign gene connected so that foreign gene can be expressed, vector, transformant, and method of producing protein

(75) Inventors: Eiichi Akahoshi, Kawasaki-shi (JP); Seiko Yoshimura, Tokyo (JP); Mitsuko Ishihara, Tokyo (JP)

Correspondence Address: Charles N.J. Ruggiero, Esq.;Ohlandt, Greeley, Ruggiero & Perle, L.L.P., One Landmark Square, 10th Floor, Stamford, CT, 06901-2682, US

(73) Assignee: KABUSHIKI KAISHA TOSHIBA

(21) Appl. No.: 11/853,536

(22) Filed: Sep. 11, 2007

(30) Foreign Application Priority Data
Sep. 29, 2006 (JP) 2006-269518

(51) Int. Cl. C12P 21/06; C12N 5/10; C12N 1/00; C12N 15/63; C07H 21/04

(52) U.S. Cl. 435/69.1; 435/325; 435/348; 435/243; 435/320.1; 536/24.1

(57) ABSTRACT

A methanol-responsive promoter includes the following regions (1) and (2) in this order in the direction from its 5′- to 3′-terminal (1) a methanol-responsive region that is selected from the group consisting of the following (a), (b) and (c) (a) a polynucleotide consisting of the nucleotide sequence of SEQ ID No 1, (b) a polynucleotide consisting of the nucleotide sequence of SEQ ID No 1 wherein one or more nucleotides are deleted, substituted or added and the polynucleotide has methanol-responsive activity, and (c) a polynucleotide which hybridizes, under a stringent condition, to a polynucleotide consisting of nucleotide sequence complementary to all or part of the nucleotide sequence of SEQ ID No 1, and which has methanol-responsive activity, and (2) a core promoter region which is the minimum region necessary for transcription initiation and which has a binding site for RNA polymerase.

### CROSS-REFERENCE TO RELATED APPLICATIONS

This application is based upon and claims the benefit of priority from prior Japanese Patent Application No. 2006-269518, filed Sep. 29, 2006, the entire contents of which are incorporated herein by reference.

### BACKGROUND OF THE INVENTION

1. Field of the Invention

The present invention relates to a methanol-responsive promoter, a fusion gene comprising the promoter and a foreign gene so connected that the foreign gene can be expressed, a vector containing the promoter, an expression vector containing the fusion gene, a transformant containing the expression vector, and a method of producing a protein using the transformant.

2. Description of the Related Art

Many valuable proteins included in biomedicines and functional foods have been produced by introducing a valuable protein-coding gene into a cell and expressing it in the cell. In production of the valuable proteins, the gene to be introduced is generally placed under the control of an inducible promoter that is capable of functioning in the cell, and its expression amount and expression period are controlled by the promoter. A methanol-responsive promoter is also one of such inducible promoters for use in production of valuable proteins. Recently, an alcohol oxidase gene promoter (AOX promoter) derived from Pichia Pastoris has been used as the methanol-responsive promoter. A desirable valuable protein has been produced by introducing a fusion gene of the AOX promoter and a foreign gene into a cell and inducing expression of the foreign gene by methanol treatment (U.S. Pat. Nos. 4,808,537, 4,855,231, 4,882,279, 4,895,800, 5,032,516, and 5,166,329).

However, in the case where a valuable protein is produced by using the AOX promoter, it has been necessary to use methanol having a relatively high concentration of 0.5% to 1% in order to induce expression of the foreign gene. Methanol is a chemical substance that is harmful to a human body and cell. Accordingly, in the case where a valuable protein is produced by using the AOX promoter, it is necessary to avoid the cytotoxic effect of methanol, and thus the kinds of cells to be used are restricted to methanol-resistant microorganisms such as Pichia Pastoris. Further, it is necessary to give care to the influence on health of a person involved in the production process using the AOX promoter.

### BRIEF SUMMARY OF THE INVENTION

According to a first aspect of the present invention, there is provided a methanol-responsive promoter comprising the following regions (1) and (2) in this order in the direction from its 5′- to 3′-terminal:

(1) a methanol-responsive region that is selected from the group consisting of the following (a), (b) and (c):

(a) a polynucleotide consisting of the nucleotide sequence of SEQ ID NO: 1,

(b) a polynucleotide consisting of the nucleotide sequence of SEQ ID NO: 1 wherein one or more nucleotides are deleted, substituted or added and the polynucleotide has methanol-responsive activity, and

(c) a polynucleotide which hybridizes, under a stringent condition, to a polynucleotide consisting of nucleotide sequence complementary to all or part of the nucleotide sequence of SEQ ID No: 1, and which has methanol-responsive activity; and

(2) a core promoter region which is the minimum region necessary for transcription initiation and which has a biding site for RNA polymerase.

According to a second aspect of the present invention, there is provided a fusion gene comprising the methanol-responsive promoter according to the first aspect and a foreign gene connected to downstream of the methanol-responsive promoter so that the foreign gene can be expressed.

According to a third aspect of the present invention, there are provided a vector comprising the methanol-responsive promoter according to the first aspect, and an expression vector comprising the fusion gene according to the second aspect.

According to a fourth aspect of the present invention, there is provided a transformant wherein the fusion gene according to the second aspect or the expression vector according to the third aspect is introduced into a host cell.

According to a fifth aspect of the present invention, there is provided a method of producing a protein, comprising: culturing the transformant according to the fourth aspect in a culture solution; treating the transformant with methanol by adding methanol to the culture solution; and recovering a protein that is a product of the foreign gene produced by the transformant, after the treatment with methanol.

### BRIEF DESCRIPTION OF THE SEVERAL VIEWS OF THE DRAWING

FIG. 1 is a graph showing the relationship between various lengths of a transcriptional regulatory region in tyrosine hydroxylase gene and methanol-responsive activity;

FIG. 2A is a schematic chart showing a method of preparing a pTH100E32/LUC expressing vector according to an embodiment;

FIG. 2B is a schematic chart showing a method of preparing a pSV40E32/LUC expressing vector according to an embodiment;

FIG. 3A is a graph showing methanol-responsive activity of the pTH100E32/LUC expressing vector according to an embodiment;

FIG. 3B is a graph showing methanol-responsive activity of the pSV40E32/LUC expressing vector according to an embodiment;

FIG. 4 is a graph showing methanol-responsive activity of the pTH100E32/LUC expressing vector and a pTH100E32×3/LUC expressing vector according to an embodiment; and

FIG. 5 is a graph showing concentration-dependent methanol-responsive activity of the pTH100E32×3/LUC expressing vector according to an embodiment.

### DETAILED DESCRIPTION OF THE INVENTION

The present inventors have found that the transcriptional regulatory region of a tyrosine hydroxylase gene shows methanol-responsive activity in responsive to low concentration of methanol and thereby completed the present invention.

[1. Methanol-Responsive Promoter]

In an embodiment of the present invention, the methanol-responsive promoter comprises:

(1) a methanol-responsive region consisting of the nucleotide sequence shown by SEQ ID No: 1; and

(2) a core promoter region, which is the minimum region needed for transcription initiation and has a binding site for RNA polymerase, in this order in the direction from its 5′- to 3′-terminal.

The methanol-responsive region shown by SEQ ID No: 1 is a region which is derived from an upstream region of a mouse tyrosine hydroxylase gene (hereinafter, also referred to as TH gene), i.e., a neurotransmitter dopamine synthesis enzyme gene, and which controls expression of the TH gene.

In an embodiment, the methanol-responsive region is a polynucleotide consisting of the nucleotide sequence shown by SEQ ID No: 1.

In another embodiment, the methanol-responsive region may be a partial sequence of the nucleotide sequence shown by SEQ ID No: 1, as long as it still shows methanol-responsive activity. It is possible to determine whether the partial sequence shows methanol-responsive activity, by incorporating the partial sequence into an expression vector containing a reporter gene and testing increased expression of the reporter gene by addition of methanol.

In yet another embodiment, the methanol-responsive region may be the following polynucleotide (b) or (c):

(b) a polynucleotide consisting of the nucleotide sequence shown by SEQ ID No: 1, whose one or more nucleotides are deleted or substituted, or to which one or more nucleotides are added, and which has methanol-responsive activity;

(c) a polynucleotide which hybridizes, under a stringent condition, to a polynucleotide consisting of a sequence complementary to all or part of the nucleotide sequence shown by SEQ ID No: 1 and which has methanol-responsive activity.

Thus, one or more (e.g., several) nucleotides may be deleted, substituted, or added in the methanol-responsive region of the nucleotide sequence shown by SEQ ID No: 1, as long as the polynucleotide shows methanol-responsive activity. Alternatively, the methanol-responsive region may be a polynucleotide that hybridizes, under a stringent condition, to a polynucleotide consisting of a sequence complementary to (all or part of) the nucleotide sequence shown by SEQ ID No: 1, as long as the polynucleotide shows methanol-responsive activity.

The polynucleotides (b) and (c) can be prepared, for example, by point mutation or hybridization method based on the sequence of SEQ ID No: 1. The stringent condition is a condition which allows the formation of so-called specific hybrid but dose not allow the formation of non-specific hybrid. For example, it is a condition which allows hybridization between nucleic acids having a higher homology, such as DNAs having a homology of 60% or more, preferably 80% or more, but inhibits hybridization between nucleic acids having a homology lower than that. More specifically, the stringent condition means that a tested nucleic acid is hybridized with a nucleic acid having a complementary sequence by means of hybridization at 42° C. and washing at approximately 42° C. with a 1×SSC buffer solution containing 0.1% SDS, or by means of hybridization at 65° C. and washing at approximately 65° C. with a 0.1×SSC buffer solution containing 0.1% SDS.

In yet another embodiment, the methanol-responsive region may be a polynucleotide derived from a TH gene transcriptional regulatory region of a species other than mouse, as long as it shows methanol-responsive activity. Thus, if a nucleotide sequence similar to the nucleotide sequence of SEQ ID No: 1 is found in the TH gene transcriptional regulatory region of other species, it may be used as the methanol-responsive region in the present invention, as long as it shows methanol-responsive activity.

As described above, the methanol-responsive region is not limited to the nucleotide sequence shown by SEQ ID No: 1, and may be a partial sequence thereof, a sequence containing some modification in the nucleotide sequence of SEQ ID No: 1, or a sequence derived from other species, as long as it shows methanol-responsive activity.

The core promoter region in the promoter according to the present invention is the minimum region needed for initiation of transcription and contains a binding site for RNA polymerase. In other words, the core promoter region is the minimum region needed for inducing initiation of transcription and maintaining at least the lowest transcription level, which consists of a nucleotide sequence surrounding the transcription initiation site including the transcription initiation point (+1). The core promoter region contains a RNA polymerase-binding site as its essential element, and generally contains a TATA box as the element important for transcription-initiating reaction.

Any one of nucleotide sequences known in the art as core promoter or minimum promoter may be used as the core promoter region. Specifically, a region ranging from −100 to +35, preferably a region ranging from −35 to +35, located in upstream region of a structure gene may be used. For example, a TH gene core promoter region consisting of the nucleotide sequence shown by SEQ ID No: 2 may be used as the core promoter region.

A known promoter sequence in which the core promoter region has not been identified may also be used as the core promoter region. In such a case, the selected promoter sequence may contain a sequence beyond the core promoter region, as long as the sequence can function as a core promoter region when connected to the methanol-responsive region. For example, a simian virus 40 (SV40) early promoter (SEQ ID No: 3), a SV40 late promoter (SEQ ID No: 4), or a human herpesvirus 1 thymidine kinase promoter (SEQ ID No: 5) may be used as the core promoter region.

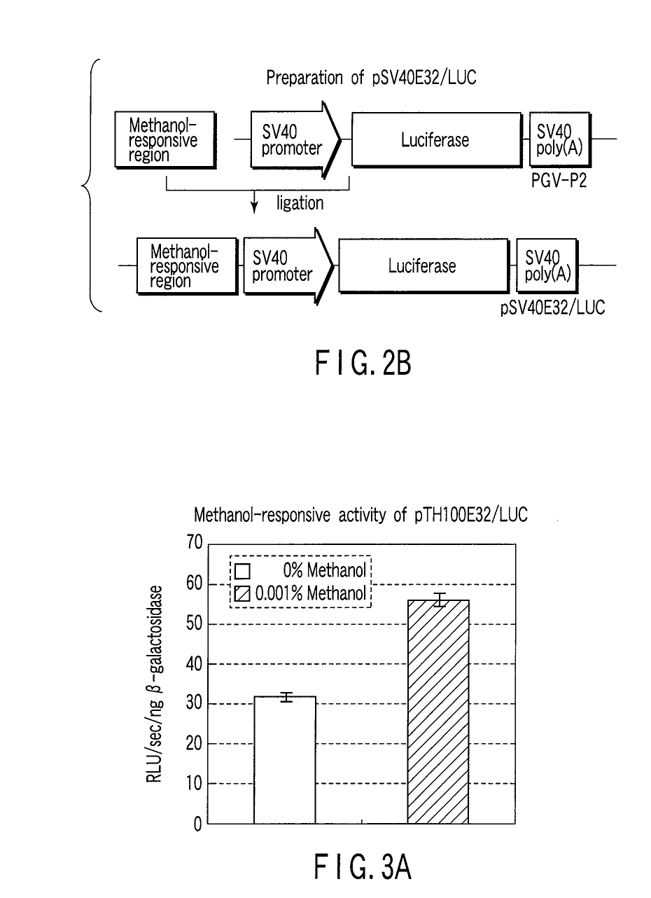

Specific examples of the methanol-responsive promoter according to the present invention include a promoter consisting of the nucleotide sequence shown by SEQ ID No: 6. It is a promoter comprising the methanol-responsive region of a mouse TH gene (SEQ ID No: 1) and the core promoter region of a mouse TH gene (SEQ ID No: 2) in this order in the direction from the 5′- to 3′-terminal (see FIG. 2A). The first to 143rd nucleotides in the sequence of SEQ ID No: 6 correspond to the methanol-responsive region, and the 171st to 260th nucleotides in the sequence of SEQ ID No: 6 correspond to the core promoter region. Another specific example of the methanol-responsive promoter is a promoter consisting of the nucleotide sequence shown by SEQ ID No: 7. It is a promoter comprising the methanol-responsive region of a mouse TH gene (SEQ ID No: 1) and a SV40 early promoter (SEQ ID No: 3) in this order in the direction from the 5′- to 3′-terminal (see FIG. 2B). The first to 143rd nucleotides in the sequence of SEQ ID No: 7 correspond to the methanol-responsive region, and the 281st to 373rd nucleotides in the sequence of SEQ ID No: 7 correspond to the core promoter region. Yet another specific example of the methanol-responsive promoter is a promoter consisting of the nucleotide sequence shown by SEQ ID No: 8. It is a promoter comprising three copies of the methanol-responsive regions of a mouse TH gene (three copies of SEQ ID No: 1) and the core promoter region of a mouse TH gene (SEQ ID No: 2) in this order in the direction from the 5′- to 3′-terminal (see Example 5). The first to 143rd nucleotides, the 156th to 298th nucleotides, and the 311th to 453rd nucleotides in the sequence of SEQ ID No: 8 correspond to the three copies of the methanol-responsive regions, and the 471st to 570th nucleotides in the sequence of SEQ ID No: 8 correspond to the core promoter region.

As shown in the specific examples above, the methanol-responsive region and the core promoter region are preferably located closer to each other, so that the methanol-responsive region can control the efficiency of the transcription-initiating reaction caused by the core promoter. Generally, the methanol-responsive promoter is prepared by ligation of these regions through a genetic engineering process, and thus it may have a connection sequence, for example a connection sequence of 4 bp to 8 bp, between these two regions.

As shown in the specific examples above, the methanol-responsive promoter may contain one methanol-responsive region, or multiple copies of the methanol-responsive regions in tandem (in series) in a preferable embodiment. Thus, multiple copies of the methanol-responsive regions may be placed continuously on the same sequence. Generally, 2 to 10 copies, preferably 2 to 5 copies, of the methanol-responsive regions may be connected to each other. The multiple copies of the methanol-responsive regions may be the same as or different from each other in the nucleotide sequence. In this manner, multiple copies of the methanol-responsive regions are placed continuously in tandem, and thereby it is possible to increase the sensitivity of methanol-responsive activity (see Example 5).

As is known in the art, the methanol-responsive promoter according to the present invention can be prepared by amplifying the methanol-responsive region by PCR using the genomic DNA of a desirable organism as a template, amplifying the core promoter region by PCR using the genomic DNA of a desirable organism as a template, and ligating them to each other.

The methanol-responsive promoter according to the present invention can also be prepared by chemical synthesis or by hybridization using a DNA fragment having the nucleotide sequence as a probe, from a genomic DNA library of a desirable organism. Further, one or more nucleotides may be deleted from the prepared methanol-responsive promoter, substituted in the prepared methanol-responsive promoter, or added to the prepared methanol-responsive promoter by site-specific mutagenesis.

It is possible to confirm whether the methanol-responsive promoter can function, by the following method. For example, it is possible to confirm the function of the promoter by preparing a vector having a reporter gene (luciferase gene (LUC), green fluorescent protein gene (GFP), or the like) connected to downstream of the promoter, introducing the vector into a host cell, and then measuring expression of the reporter gene caused by methanol treatment.

As described above, the methanol-responsive promoter according to the present invention can be used as an inducible promoter, whose promoter activity can be induced in response to a low concentration of methanol. As will be shown in Example 6 described below, the methanol-responsive promoter according to the present invention can enhance expression of its downstream genes, in response to a low concentration of methanol, for example, 0.001 to 0.1 vol % of methanol. Therefore, in the present invention, it is possible to effectively produce a valuable protein coded by a gene that is placed under the control of the methanol-responsive promoter according to the present invention, by treatment with a low concentration of methanol.

The methanol-responsive promoter according to the present invention has an advantage that a host cell used for gene expression is not limited to a special cell such as a methanol-resistant microorganism, for the reason that it can respond to a low concentration of methanol. In addition, it also has an advantage that it is possible to reduce the adverse effects of methanol on those who are involved in producing the valuable protein.

In the present invention, it is also possible to carry out a gene expression in response to methanol in any host cell. Specifically, it is possible to carry out a gene expression in response to methanol in any host cell by selecting a core promoter region functionable in a desirable host cell, connecting it to the above mouse TH gene-derived methanol-responsive region, and then, preparing a chimera promoter.

Function of such a chimera promoter as a methanol-responsive promoter will be verified in Example 4 described below.

[2. Fusion Gene of the Promoter and Foreign Gene]

A fusion gene of the above methanol-responsive promoter and a foreign gene can be prepared by connecting the foreign gene to a region downstream of the methanol-responsive promoter, so that the foreign gene can be expressed, according to a method known in the art. Expression of the foreign gene is enhanced under the control of the methanol-responsive promoter. The phrase “so that the foreign gene can be expressed” herein means that the foreign gene is so connected to the promoter that it can be expressed under the control of the promoter. For example, it means that the foreign gene is connected to the promoter via a gene insertion site downstream of the promoter (e.g., a multicloning site).

The foreign gene may be any known gene, and examples thereof include a protein-coding nucleic acid, an antisense nucleic acid thereof, and the like, and, among them, a nucleic acid coding a desired valuable protein are preferable. Examples of the nucleic acid coding a valuable protein include a gene coding a vaccine protein such as cholera toxin protein variant (Yamamoto, S. et al., J. Exp. Med., 185: 1203, 1997; Douce, G. et al., Proc. Natl. Acad. Sci. USA., 92: 1644, 1995; Di Tommaso, A. Infect. Immun., 64: 974, 1996; Dickinson, B. L. et al., Infct. Immun., 63: 1617, 1995), a gene coding an antibody protein such as anti-herpes simplex virus IgG gene (Zeitlin L. et al., Nat. Biotechnol., 16: 1361, 1998), a gene coding a pharmaceutical protein such as collagen gene (JP-A 2004-016144 (KOKAI)) or lactoferrin gene (JP-A 2001-346577 (KOKAI)), and the like.

[3. Vector Containing the Methanol-Responsive Promoter, and Expression Vector Containing the Fusion Gene of the Promoter and Foreign Gene]

Hereinafter, the vector containing the above methanol-responsive promoter (hereinafter, also referred to as the vector according to the present invention), and the expression vector containing the above fusion gene of the promoter and a foreign gene (hereinafter, also referred to as the expression vector according to the present invention) will be described.

A commercially available vector may be used as an original vector in the preparation of the vector according to the present invention and the expression vector according to the present invention. Specifically, the original vector for insertion of the promoter according to the present invention is not particularly limited, as long as it is a plasmid vector or a chromosome-integrating vector that is integrated into the genome of a host organism, and examples thereof include plasmid DNA, viral DNA, and the like. Examples of the plasmid DNA include an Escherichia coli-yeast shuttle vector, an Escherichia coli-derived plasmid, and the like, and examples of the viral DNA include an animal viral vector such as retrovirus and vaccinia virus, an insect viral vector such as baculovirus, and the like.

The vector according to the present invention can be obtained by inserting the above methanol-responsive promoter into a suitable vector by a known method, and the expression vector according to the present invention can be obtained by inserting the above fusion gene of the promoter and a foreign gene into a suitable vector by a known method. Specifically, the vector and the expression vector according to the present invention can be prepared by purifying a DNA to be inserted, digesting the purified DNA with a suitable restriction enzyme, and inserting the obtained DNA into a restriction site or a multicloning site of an original vector.

The vector according to the present invention preferably has a multicloning site for incorporation of a foreign gene in the region downstream of the methanol-responsive promoter.

In addition to the methanol-responsive promoter, a foreign gene, and a terminator, a splicing signal, a poly A addition signal, a selective marker, or the like may be incorporated into the vector and the expression vector according to the present invention. Examples of the selective marker include an antibiotic resistance gene such as ampicillin resistance gene, hygromycin resistance gene, and zeocin resistance gene.

In the Examples below, expression vectors “pTH100E32/LUC” and “pSV40E32/LUC” (Example 2) and expression vectors “pTH100E32×3/LUC” containing three copies of the methanol-responsive regions (Example 5) are prepared by using commercially available vectors PGV-B2 or PGV-P2 (available from TOYO B-NET) as an original vector. In the Examples, the expression vectors pTH100E32/LUC and pSV40E32/LUC are prepared according to the procedures schematically shown in FIGS. 2A and 2B, respectively.

[4. Transformant Having the Fusion Gene of the Promoter and Foreign Gene or the Expression Vector Introduced into Host Cell]

The transformant according to the present invention can be obtained by introducing the fusion gene of the promoter and a foreign gene or the expression vector according to the present invention into a suitable host cell by a known method.

The host cell may be selected according to the type of the core promoter region contained in the introduced methanol-responsive promoter. In other words, a host cell allowing expression of a foreign gene located downstream of the methanol-responsive promoter may be selected.

Examples of the host cell include, but are not limited to, bacteria such as Escherichia coli, Bacillus, and Pseudomonas; yeast such as Saccharomyces, Schizosaccharomyces, and Pichia; animal cells such as Neuro2a, COS cell and CHO cell; and insect cells such as Sf9.

When a bacterium such as Escherichia coli is used as the host, an introduced vector is preferably autonomously replicable in the bacterium, and preferably has a ribosome-binding sequence, the promoter of the present invention, and a transcription termination sequence. The method of introducing the vector into a bacterium is not particularly limited, as long as it allows introduction of DNA into the bacterium. Examples thereof include a method of using calcium ion, electroporation, and the like.

When yeast is used as the host, Saccharomyces, Schizosaccharomyces, Pichia, or the like may be used. The method of introducing a vector into yeast is not particularly limited, as long as it allows introduction of DNA into the yeast, and examples thereof include electroporation, a spheroplast method, a lithium acetate method, and the like.

When an animal cell is used as the host, simian COS-7 cell, Chinese hamster CHO cell, mouse L cell, mouse Neuro2a cell, rat PC12 cell, human FL cell, or the like may be used. Examples of the method of introducing a vector into an animal cell include electroporation, a calcium phosphate method, a lipofection method, and the like.

When an insect cell is used as the host, Sf9 cell or the like may be used. Examples of the method of introducing a vector into an insect cell include a calcium phosphate method, a lipofection method, electroporation method, and the like.

In the Examples below, transformants are prepared by introducing any one of expression vectors “pTH100E32/LUC”, “pSV40E32/LUC” and “pTH100E32×3/LUC” into a mouse-derived neuroblastoma by a Lipofectamine method (Examples 3 and 5).

[5. Method of Producing Protein by Expressing Gene Under the Control of the Methanol-Responsive Promoter]

In an embodiment of the present invention, the method of producing a protein comprises: a step of culturing the above transformant in a culture solution; a step of treating the transformant with methanol by adding methanol to the culture solution; and a step of recovering a foreign gene product protein produced by the transformant, after the methanol treatment.

The transformant is cultured in a suitable culture solution with the aim of producing a protein from an introduced gene. Escherichia coli, yeast, or an insect cell is preferable as the host cell used for the production of a protein, from the viewpoint of convenience in culturing. As the culture solution, LB medium, 2xYT medium, or Super broth medium may be used when the transformant is Escherichia coli; YPD medium or BMMY medium may be used, when it is a yeast cell; and Sf900 medium or TC100 medium may be used when it is an insect cell. The culture condition (culture temperature, culture period, and others) of the transformant is not particularly limited, and any conventional culture condition may be used.

Methanol is added at a promoter-activity enhancing concentration, generally at a concentration of 0.001 to 0.5 vol %, preferably 0.001 to 0.1 vol %, to the culture solution. Addition of methanol causes increase in expression of a gene under the control of the methanol-responsive promoter and thereby protein production. In the present specification, the concentration of methanol means the final concentration in the cell culture solution.

The protein produced from the foreign gene expression can be collected, for example, 24 to 72 hours after the addition of methanol. The produced protein can be collected according to a known protein purification method, for example, by column chromatography, salting out, electrophoresis, gel filtration, or the like.

### EXAMPLES

### Example 1

### Identification of “Methanol-Responsive Promoter Region” in Tyrosine Hydroxylase (TH) Gene

The transcriptional regulatory region (approximately 500 bp) of tyrosine hydroxylase (TH) was amplified by PCR, by using a mouse genome previously digested with a restriction enzyme KpnI (Clontech) as a template. As the primers, the following primers were used for facilitating incorporation of the PCR products into a vector: the primer of SEQ ID No: 9 (forward primer) having the recognition sequence for restriction enzyme KpnI added to the 5′ terminal, and the primer of SEQ ID No: 10 (reverse primer) having the recognition sequence for NheI added to the 5′ terminal.

(SEQ ID No: 9)

5′-CGTGGTACCA CATACACTGG GGCAGTGAGT AGAT-3′

(SEQ ID No: 10)

5′-GCAGCTAGCA AGCTGGTGGT CCCGAGTTCT GTCT-3′

Then, four kinds of TH transcriptional regulatory region fragments having 400 to 100 bp in length at an interval of approximately 100 bp were prepared by PCR using the amplified TH transcriptional regulatory region (500 bp) as the template. Any one of the primers shown by SEQ ID Nos: 11, 12, 13, and 14 was used as the forward primer, and the primer of SEQ ID No: 10 was used as the reverse primer in the PCR.

(SEQ ID No: 11)

For 400 bp fragment: 5′-CGGGGTACCA GATTTATTTG
TCTCCAAGGG CTAT-3′

(SEQ ID No: 12)

For 300 bp fragment: 5′-CGGGGTACCA TTAGAGAGCT
CTAGATGTCT CCTG-3′

(SEQ ID No: 13)

For 200 bp fragment: 5′-CCCGGTACCC TAATGGGACG
GAGGCCTCTC TCGT-3′

(SEQ ID No: 14)

For 100 bp fragment: 5′-CGGGGTACCG TGGGGGACCC
AGAGGGGCTT TGAC-3′

Then, the five kinds of TH gene transcriptional regulatory region different in length (500 bp, 400 bp, 300 bp, 200 bp and 100 bp) were digested with KpnI and NheI and subjected to 0.8% agarose gel electrophoresis. Thereafter, the five kinds of DNA fragments were excised from the gel and purified with QIA quick Gel Extraction Kit (QIAGEN). Each of the purified DNA fragments was inserted into PGV-B2 vector (available from TOYO B-NET) previously digested with KpnI and NheI, thereby preparing luciferase expressing vectors containing any one of the DNA fragments (500 bp, 400 bp, 300 bp, 200 bp and 100 bp).

Each of the vectors thus prepared was transfected into mouse neuroblastoma Neuro2a by Lipofectamine method, thereby obtaining transfected cells having any one of the DNA fragments (500 bp, 400 bp, 300 bp, 200 bp and 100 bp). The “methanol-responsive promoter region” of TH gene was identified by using the obtained five types of transfected cells. Specifically, 0.4 μg of the luciferase expressing vector and 0.4 μg of a galactosidase expressing vector (pcDNA4/V5-His/LacZ, Invitrogen) were suspended in 50 μl of Opti-MEM medium; an Opti-MEM medium containing 2 μl of Lipofectamine 2000 (Invitrogen) was added thereto; and the mixture was left at room temperature for 20 minutes to form a nucleic acid/Lipofectamine 2000 complex. Then, the complex was added to Neuro2a cell, which had been previously prepared by culturing overnight in Dulbecco/Ham's mixed medium (DF1:1 medium) containing 10% fetal calf serum (by seeding it in the medium on the previous day at a concentration of 0.8×10⁵ cells).

After 24 hours, the transfected Neuro2a cell (having any one of the 100 to 500 bp of TH gene transcriptional regulatory regions) was exposed to 1% methanol, and the methanol-responsive activity of the TH gene transcriptional regulatory region was determined based on the change in expression of the reporter gene.

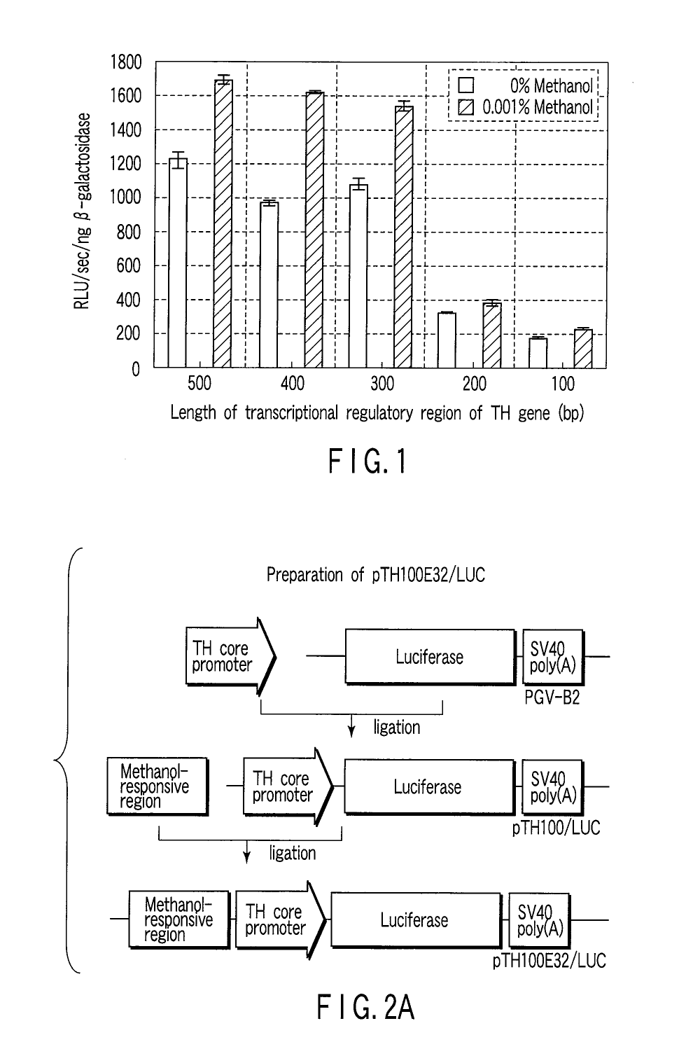

The results are summarized in FIG. 1. As shown in FIG. 1, methanol-responsive activity was confirmed in the case of the luciferase expressing vector containing the TH gene transcriptional regulatory region of 500 bp, 400 bp, or 300 bp in length, but there was no methanol-responsive activity in the case of the luciferase expressing vector containing the same region of 200 bp or 100 bp. This indicates that the upstream region located in approximately 300 to 200 bp upstream of TH gene is involved in the methanol-responsive activity of TH gene. That is, the results show that the upstream region located in approximately 300 to 200 bp upstream of TH gene is essential for maintaining the methanol-responsive activity of the promoter according to the present invention.

### Example 2

### Preparation of Methanol-Responsive Promoter and Luciferase Expressing Vector Containing the Methanol-Responsive Promoter

A genomic DNA extracted from a mouse was digested with KpnI, and approximately 500 base pairs of the upstream region of the tyrosine hydroxylase (TH) gene was amplified by PCR using the digested genomic DNA as the template and the nucleotide sequences shown by SEQ ID Nos: 15 and 10 as the primers. The amplified DNA fragment was subjected to 0.8% agarose gel electrophoresis, the DNA fragment were excised from the gel and purified with QIA quick Gel Extraction Kit (QIAGEN).

(SEQ ID No: 15)

5′-CGGCTCGAGG TGGGGGACCC AGAGGGGCTT TGAC-3′

Then, approximately 100 bp (SEQ ID No. 2) of the upstream region of TH gene was amplified by PCR using the obtained approximately 500 bp of upstream region of TH gene as a template and the nucleotide sequences shown by SEQ ID Nos: 12 and 16 as the primers. The amplified DNA fragment was digested with XhoI and HindIII.

(SEQ ID No: 16)

5′-GCCGCTAGCA CGAGAGAGGC CTCCGTCCCA TTAG-3′

The DNA fragment digested was subjected to 0.8% agarose gel electrophoresis, and the DNA fragment was excised from the gel and purified with QIA quick Gel Extraction Kit (QIAGEN). The purified DNA fragment (core promoter region) was ligated with the vector PGV-B2 (TOYO B-Net) digested with XhoI and HindIII, by using a ligation kit (Toyobo). The resultant product was introduced into E. coli TOP10 strain (Invitrogen).

Ampicillin resistance strains were selected from the E. coli TOP10 strains thus prepared. Plasmid DNA was collected from the selected ampicillin resistance strains, thereby obtaining a “pTH100/LUC expressing vector” (i.e., a vector containing the nucleotide sequence shown by SEQ ID No: 2 incorporated in the PGV-B2 vector) (see FIG. 2A).

Further, the region shown by SEQ ID No: 1 was amplified by PCR using the above approximately 500 bp of the upstream region of TH gene as a template and the nucleotide sequences shown by SEQ ID Nos: 17 and 18 as primers. The amplified DNA (methanol-responsive region) was digested with KpnI and NheI. The resultant product was subjected to 0.8% agarose gel electrophoresis, and the DNA fragment was excised from the gel and purified with QIA quick Gel Extraction Kit (QIAGEN).

(SEQ ID No: 17)

5′-CGGGGTACCA TTAGAGAGCT CTAGATGTCT CCTG-3′

(SEQ ID No: 18)

5′-GCCGCTAGCA CGAGAGAGGC CTCCGTCCCA TTAG-3′

The purified nucleotide sequence shown by SEQ ID No: 1 was ligated with the pTH100/LUC expressing vector digested similarly with KpnI and NheI, by using a ligation kit (Toyobo). The resultant product was introduced into E. coli TOP10 strain (Invitrogen). Ampicillin resistance strains were selected from the E. coli TOP10 strains thus prepared. Plasmid DNA was collected from the selected ampicillin resistance strains, thereby obtaining a “pTH100E32/LUC expressing vector” (i.e., a vector containing the nucleotide sequence shown by SEQ ID No: 1 incorporated in the pTH100/LUC expressing vector) (see FIG. 2A).

On the other hand, the purified nucleotide sequence shown by SEQ ID No: 1 was ligated with the vector PGV-P2 (TOYO B-NET) digested with KpnI and NheI, by using a ligation kit (Toyobo). The resultant product was introduced into E. coli TOP10 strain (Invitrogen). The used vector PGV-P2 contains a SV40 early promoter (SEQ ID No: 3) previously as the core promoter region. Ampicillin resistance strains were selected from the E. coli TOP10 strains thus prepared. Plasmid DNA was collected from the selected ampicillin resistance strains, thereby obtaining a “pSV40E32/LUC expressing vector” (i.e., a vector having the nucleotide sequence shown by SEQ ID No: 1 incorporated in the PGV-P2 vector) (see FIG. 2B).

### Example 3

### Introduction of pTH100E32/LUC Expressing Vector into Mammal Cell, and Introduction of pSV40E32/LUC Expressing Vector into Mammal Cell

The pTH100E32/LUC expressing vector and the pSV40E32/LUC expressing vector described in Example 2 were introduced respectively into a mouse-derived neuroblastoma Neuro2a strain. They were introduced by the Lipofectamine method. Specifically, a total of 0.8 μg of the vectors to be introduced (luciferase expressing vector (either pTH100E32/LUC or pSV40E32/LUC) and P-galactosidase expressing vector (pcDNA4/V5-His/LacZ)) was suspended in 50 μl of Opti-MEM medium, and 2 μl of Lipofectamine 2000 (Invitrogen) was suspended in 50 μl of Opti-MEM medium. They were mixed and the mixture was left at room temperature for 20 minutes to form a nucleic acid/Lipofectamine 2000 complex. Then, the complex was added to Neuro2a cell, which had been previously prepared by culturing overnight in Dulbecco/Ham's mixed medium (DF1:1 medium) containing 10% fetal calf serum (by seeding it in the medium on the previous day at a concentration of 0.8×10⁵ cells). As a result, the vectors were transfected into the Neuro2a cell.

In the above, two kinds of expression vectors, i.e., a luciferase expressing vector (either pTH100E32/LUC or pSV40E32/LUC) and β-galactosidase expressing vector (pcDNA4/V5-His/LacZ) (Invitrogen), were transfected into the cell. The pcDNA4/V5-His/LacZ was transfected for standardization of the vector-introducing efficiency. After the transfection, the cell containing the expression vectors was cultured additionally for 24 hours, for the production of luciferase protein from the vector.

### Example 4

### Methanol-Responsive Activity of pTH100E32/LUC Expressing Vector and pSV40E32/LUC Expressing Vector

The cell containing expression vectors (either pTH100E32/LUC or pSV40E32/LUC, and pcDNA4/V5-His/LacZ), which had been prepared according to the method described in Example 3, was cultured in DF1:1 medium containing 0.001 vol % of methanol for 24 hours. After 24 hours, the medium was removed, and the cell was washed twice with phosphate-buffered saline (PBS). Then, 100 μl of a protein extraction solution (TOYO B-Net) was added to each well, and the mixture was stirred gently at room temperature for 15 minutes and then frozen at −80° C. The frozen solution was thawed at room temperature and transferred into a 1.5-ml tube, and the luciferase and β-galactosidase activities thereof were measured. The luciferase activity was represented by a relative luminescence intensity per second per 1 ng of β-galactosidase per second (RLU/sec/ng β-galactosidase).

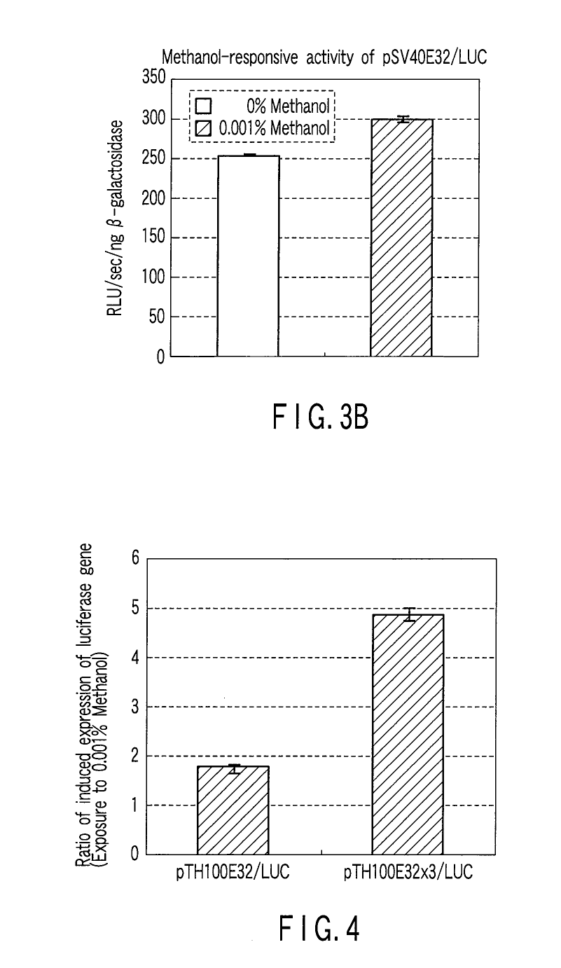

Results are summarized in FIGS. 3A and 3B. As shown in FIGS. 3A and 3B, luciferase activity increased by the treatment with 0.001 vol % of methanol in both cases of the expression vector pTH100E32/LUC and the expression vector pSV40E32/LUC.

### Example 5

### Enhancement of Methanol-Responsive Activity of pTH100E32/LUC Expressing Vector

A luciferase expressing vector containing three copies of the methanol-responsive regions of TH gene was prepared for the purpose of further enhancement of the methanol-responsive activity of the pTH100E32/LUC expressing vector.

The DNA fragment shown by SEQ ID No: 1, which corresponds to the methanol-responsive region of TH gene, was amplified by PCR using the nucleotide sequences shown by SEQ ID Nos: 17 and No. 18 as primers, as described in Example 2. Then, the 5′-terminal of the amplified DNA fragment was phosphorylated by enzyme reaction with a polynucleotide kinase. The phosphorylated DNA fragments were ligated in tandem with each other by Ligation High (TOYOBO), thereby obtaining the tandem repeat of the methanol-responsive region. The obtained tandem repeat was subjected to partial digestion with KpnI and NheI, and then to 0.8% agarose gel electrophoresis for fractionation by molecular weight. A fragment consisting of three copies of the nucleotide sequences shown by SEQ ID No: 1 was excised from the gel and purified with QIA quick Gel Extraction Kit (Qiagen); the purified fragment was digested with KpnI and NheI; and the resultant product was inserted into the pTH100/LUC expressing vector similarly digested with KpnI and NheI (see FIG. 2A). In this way, a “pTH100E32×3/LUC expressing vector” having three copies of the nucleotide sequences shown by SEQ ID No: 1 was prepared.

Then, the prepared luciferase expressing vector was transfected into mouse neuroblastoma Neuro2a according to the method described in Example 3, and the methanol-responsive activity thereof was measured by luciferase assay.

Results are summarized in FIG. 4. As shown in FIG. 4, the pTH100E32×3/LUC expressing vector showed a methanol-responsive activity higher than that of the pTH100E32/LUC expressing vector having only one nucleotide sequence shown by SEQ ID No: 1.

### Example 6

### Concentration-Dependent Methanol-Responsive Activity of pTH100E32×3/LUC Expressing Vector

The pTH100E32×3/LUC expressing vector was introduced into mouse neuroblastoma Neuro2a according to the method described in Example 3, and the resultant cell was exposed to methanol at different concentrations. After that, the concentration dependency of the methanol-responsive activity was examined by luciferase assay.

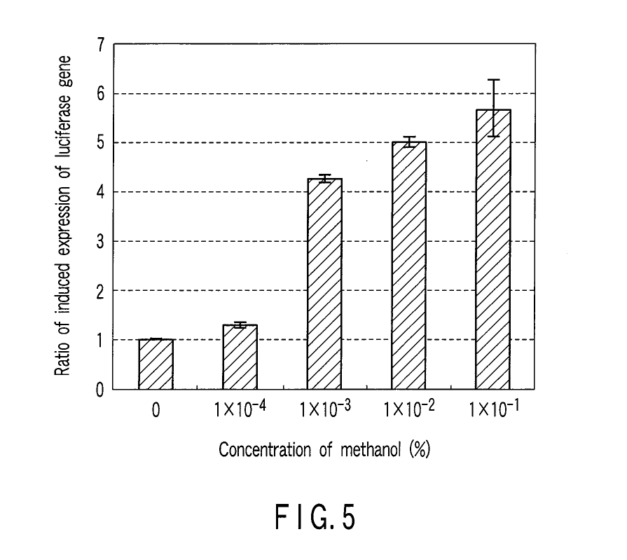

Results are summarized in FIG. 5. As shown in FIG. 5, expression of luciferase gene was induced by exposure to methanol at a concentration of 0.001 vol % or more in the cell containing the pTH100E32×3/LUC expressing vector. However, there was no significant difference in the ratio of induced expression of the reporter gene in the range of 0.001 to 0.1 vol % of methanol concentration.

What is claimed is:

1. A methanol-responsive promoter comprising the following regions (1) and (2) in this order in the direction from its 5′- to 3′-terminal:
(1) a methanol-responsive region that is selected from the group consisting of the following (a), (b) and (c):
(a) a polynucleotide consisting of the nucleotide sequence of SEQ ID No: 1,
(b) a polynucleotide consisting of the nucleotide sequence of SEQ ID No: 1 wherein one or more nucleotides are deleted, substituted or added and the polynucleotide has methanol-responsive activity, and
(c) a polynucleotide which hybridizes, under a stringent condition, to a polynucleotide consisting of nucleotide sequence complementary to all or part of the nucleotide sequence of SEQ ID No: 1, and which has methanol-responsive activity; and
(2) a core promoter region which is the minimum region necessary for transcription initiation and which has a binding site for RNA polymerase.

2. The methanol-responsive promoter according to claim 1, wherein the core promoter region is a region ranging from −100 to +35 in upstream region of a structural gene.

3. The methanol-responsive promoter according to claim 1, wherein the core promoter region is a region consisting of any one of the nucleotide sequence of SEQ ID Nos: 2 to 5.

4. The methanol-responsive promoter according to claim 1, wherein the methanol-responsive promoter comprises plurality of the methanol-responsive regions in tandem.

5. A methanol-responsive promoter consisting of any one of the nucleotide sequence of SEQ ID Nos: 6 to 8.

6. A fusion gene comprising the methanol-responsive promoter according to claim 1 and a foreign gene connected to downstream of the methanol-responsive promoter so that the foreign gene can be expressed.

7. A vector comprising the methanol-responsive promoter according to claim 1.

8. The vector according to claim 7, comprising a multicloning site in downstream of the methanol-responsive promoter.

9. An expression vector comprising the fusion gene according to claim 6.

10. A transformant wherein the fusion gene according to claim 6 is introduced into a host cell.

11. A transformant wherein the expression vector according to claim 9 is introduced into a host cell.

12. The transformant according to claim 10, wherein the host cell is selected from the group consisting of a microorganism cell, an insect cell and a mammalian cell.

13. The transformant according to claim 11, wherein the host cell is selected from the group consisting of a microorganism cell, an insect cell and a mammalian cell.

14. A method of producing a protein, comprising:
culturing the transformant according to claim 10 in a culture solution,
treating the transformant with methanol by adding methanol to the culture solution, and
recovering a foreign gene product protein produced by the transformant, after the treatment with methanol.

15. A method of producing a protein, comprising:
culturing the transformant according to claim 11 in a culture solution,
treating the transformant with methanol by adding methanol to the culture solution, and
recovering a foreign gene product protein produced by the transformant, after the treatment with methanol.
